(19) United States
(12) Patent Application Publication
(10) Pub. No.: US 2008/0199458 A1
(43) Pub. Date: Aug. 21, 2008

(54) INFLUENZA PREVENTION AND TREATMENT COMPOSITION

(76) Inventors: Jian-Er LIN, Hong Kong (CN); Wei Dong Xie, Hong Kong (CN); Kee Hung Chu, Hong Kong (CN)

Correspondence Address: EVAN LAW GROUP LLC, 600 WEST JACKSON BLVD., SUITE 625, CHICAGO, IL, 60661, US

(21) Appl. No.: 12/015,177

(22) Filed: Jan. 16, 2008

Related U.S. Application Data
(60) Provisional application No. 60/885,729, filed on Jan. 19, 2007.

(51) Int. Cl. A61K 36/48; A61K 38/16; A61K 39/395; A61K 36/06; A61K 31/045; A61K 31/075; A61K 31/015; A61K 31/215; A61K 31/351; A61K 31/195; A61P 31/12

(52) U.S. Cl. 424/130.1; 424/757; 514/12; 424/195.16; 424/730; 514/721; 514/766; 514/529; 514/459; 514/662

(57) ABSTRACT

A composition for influenza treatment includes an immune enhancer and an anti-viral agent

The present application claims priority to U.S. Provisional Application Ser. No. 60/885,729, entitled “An Influenza Prevention and Treatment Composition,” which was filed on Jan. 19, 2007, the content of which is incorporated herein by reference.

### BACKGROUND

Influenza, or commonly known as flu, is a contagious respiratory illness caused by influenza viruses. Influenza spreads around the world in seasonal epidemics, killing millions of people in pandemic years and hundreds of thousands in non-pandemic years. Typically, influenza is transmitted from infected mammals through the air and from infected birds through their droppings. Moreover, influenza infects many animal species, and transfer of viral strains between species can occur. Birds are thought to be the main animal reservoirs of influenza viruses.

Ways to prevent or inactivate influenza viruses in humans may include vaccination and anti-viral medications. An influenza vaccine is recommended for high-risk groups, such as children and the elderly, but the effectiveness of those influenza vaccines is variable. Due to the high mutation rate of the virus, a particular influenza vaccine usually confers protection for no more than a few years. Moreover, it is possible to get vaccinated and still get influenza. Anti-viral medication is sometimes effective, but viruses can develop resistance to even the standard anti-viral drugs, such as amantadine, rimantadine, zanamavir and oseltamivir.

Consequently, developing flu preventing and treating products with high efficacy are desirable. New and effective compositions are needed to prevent and treat the influenza viruses including avian flu viruses.

### BRIEF SUMMARY

According to one aspect, a composition for influenza treatment includes an immune enhancer and an anti-viral agent.

According to another aspect, a method of treating influenza includes administrating to a mammal the influenza prevention and treatment composition.

### BRIEF DESCRIPTION OF THE DRAWINGS

FIG. 1 depicts the efficacy of the composition to protect against H9N1 virus challenge in mice.

FIG. 2 depicts the efficacy of the composition of FIG. 1 compared to the composition without either an immune enhancer or an anti-viral agent.

FIG. 3 depicts the efficacy of the composition to induce TNF-α secretion.

### DETAILED DESCRIPTION

Reference will now be made in detail to a particular embodiment of the invention, examples of which are also provided in the following description. Exemplary embodiments of the invention are described in detail, although it will be apparent to those skilled in the relevant art that some features that are not particularly important to an understanding of the invention may not be shown for the sake of clarity.

Furthermore, it should be understood that the invention is not limited to the precise embodiments described below and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims. In addition, improvements and modifications which may become apparent to persons of ordinary skill in the art after reading this disclosure, the drawings, and the appended claims are deemed within the spirit and scope of the present invention.

A composition for influenza prevention and treatment includes an immune enhancer and an anti-viral agent. The immune enhancer may be used to enhance or activate the immunity of a mammal against a flu virus. The immune enhancer may include one or more agents that increase the immunity of a mammal under in-vitro, in-vivo animal or human testing. In one example, the immune enhancer is a bio-transformed soya, which is produced by Ultra Biotech Limited (a wholly owned subsidiary of CK Life Sciences Int'l. (Holdings), Inc.) in Hong Kong, China. In another example, the immune enhancer is an immune enhancing ingredient, such as lactoferrin and immunoglobulins, lingzhi, cordyceps, tinospora ingredient, or combinations thereof. The concentration of the immune enhancer in the composition may range from 1 to 99 weight percent (wt %), but preferably from 5 to 95 wt %.

The anti-viral agent of the composition is configured to inactivate the function, reduce or inhibit the effectiveness of an invading flu virus, and thus may include any agent that has that effect on the flu viruses. For example, the anti-viral agent may be St. John's wort, which is an extract or any part of the Hypericum perforatum plant, or a purified compound of the Hypericum perforatum plant, or a man-made synthesized compound of the Hypericum perforatum plant, such as hypericin and pseudohypericin. Other examples of the anti-viral agents may include adamantane, oseltamivir, zanamivir, amantadine and rimantadine.

The concentration of the anti-virus agent in the composition may range from 1 to 99 wt %, but preferably from 5 to 95 wt %. In one example, the anti-viral agent includes St. John's wort, which is standardized to contain 0.3% w/w of hypercin, from about 20 mg to 5,000 mg per daily dosage. In another example, the anti-viral agent includes St. John's wort from about 100 mg to 2,000 mg per daily dosage.

The specific concentrations of the immune enhancer and anti-virus agent may vary depending on the use. In one example, the composition may include 12.5 wt % of the immune enhancer and 87.5 wt % of the anti-virus agent. In another example, the composition may include 45 wt % of the immune enhancer and 55 wt % of the anti-virus agent. Other inert ingredients, such as filler and flavoring agent, may also be incorporated into the composition.

The function of the composition may include protecting a subject from being infected by a virus, minimizing the effect of an invading flu virus when and if a subject is infected, and accelerating the recovery from a virus infection when and if a subject is infected. In particular, the composition may be used in humans or mammals to prevent the infection of the flu and to treat flu symptoms after a viral infection, including avian flu viruses of H9N1 and H5N1.

The composition may be in a tablet, capsule or liquid form, and may be adapted for oral administration, injections and other methods.

### EXAMPLES

### Example 1

### In-Vitro Tests—Anti-Flu Virus (H9N1)

Flu virus, H9N1, was added into each of the 96-well plate at the initial concentration of 0.5, 5, 50 and 500 plague formation units per well (or pfu/well), respectively. The composition for influenza prevention and treatment was then dissolved in 2% serum-containing DMEM medium, and added into the wells at 8, 40 and 200 ppm, respectively. The medium without the composition was used as a control. The plates were incubated at 37° C. and at 5% CO₂ for three days. The H9N1 virus counts were recorded.

The composition for influenza prevention and treatment was prepared by mixing 87.5% (w/w) St. John's wort (hypericin content:0.33%) and 12.5% (w/w) bio-transformed soy (from Ultra Biotech Limited). The St. John's wort was used as the anti-virus agent and the bio-transformed soy as the immune enhancer. This composition was used for all the tests shown in all the following examples except otherwise indicated.

The results are summarized in Table 1 and displayed below. As shown, when the initial H9N1 concentration was 500 pfu/well and received no treatment, the H9N1 concentration increased substantially after three days, denoted as too numerous to count (TNTC), which indicated that the viruses populated. Comparatively, under the same initial conditions but when the composition was administered, the H9N1 concentration significantly decreased after a three-day treatment, which indicated that most of the viruses lost the viability. At a higher concentration of the administered composition, a lower H9N1 concentration was observed after a three-day treatment, hence suggesting that at higher concentrations of the composition, more viruses had lost their viability. Similar trends were also observed when the initial H9N1 concentrations were at 50 and 5 pfu/well. Consequently, these results show that the composition has an anti-viral efficacy towards H9N1, with higher concentrations of the composition exhibiting higher efficacy.

TABLE 1
In-vitro results of anti-virus (H9N1) effect of the composition

| | |
|---|---|
| Initial | |
| H9N1 | H9N1 conc. (pfu/well) after 3-day |
| conc. | treatment with the composition at: |

| (pfu/well) | 200 ppm | 40 ppm | 8 ppm | 0 ppm |
|---|---|---|---|---|
| 500 | 32 | 147 | 176 | TNTC |
| 50 | 1 | 6 | 7 | 21 |
| 5 | 0 | 0 | 0 | 2 |
| 0.5 | 0 | 0 | 0 | 0 |

### Example 2

### Flu Virus (H9N1) Tests in Chicken Embryos

A saline solution was used to dilute the composition for influenza prevention and treatment to 8, 200 and 1000 ppm, respectively. The H9N1 virus (at 10e-4) was mixed with different concentrations of the composition at a ratio of 1:9, and inoculated into the chicken embryos at a rate of 0.2 ml per embryo. For the virus control, the saline solution was used in the place of the composition solution. In addition, only the saline solution without H9N1 virus was incubated into the embryos as the non-challenged control. All the samples were then incubated at 37° C. and observed for embryo viability.

The results were displayed in Table 2. When the chicken embryos were presented the H9N1 virus challenge and received no treatment, about 75 percent of them had lost their viability by day 3 and all of them were not viable by day 4. This represented the virus control. When the chicken embryos were presented the H9N1 virus challenge and received 8 ppm of the composition, only about 34 percent of them had lost their viability by day 3. When the chicken embryos were presented the H9N1 virus challenge and received 200 ppm of the composition, only about 34 percent of them had lost the viability by day 6. When the chicken embryos were presented the H9N1 virus challenge and received 1000 ppm of the composition, all of them were still alive by day 8. When the chicken embryos were not presented the H9N1 virus challenge and no treatment, all of them were also still alive by day 8.

TABLE 2
Protection against H9N1 Virus Challenge in Chicken Embryos

| | Percent embryo survival at | | | | | | | |
|---|---|---|---|---|---|---|---|---|
| Treatments | Day 0 | Day 1 | Day 2 | Day 3 | Day 4 | Day 5 | Day 6 | Day 7 |
| 1000 ppm composition | 100 | 100 | 100 | 100 | 100 | 100 | 100 | 100 |
| 200 ppm composition | 100 | 100 | 100 | 100 | 100 | 100 | 66.7 | 66.7 |
| 8 ppm composition | 100 | 100 | 100 | 66.7 | 66.7 | 66.7 | 66.7 | 66.7 |
| Virus control | 100 | 100 | 25 | 0 | 0 | 0 | 0 | 0 |
| Non-challenged control | 100 | 100 | 100 | 100 | 100 | 100 | 100 | 100 |

As seen, at higher concentrations of the composition treatment, more chicken embryos had lived. Consequently, the results show that the composition can protect the chicken embryos against H9N1 virus, with higher concentration of the composition demonstrating higher efficacy.

### Example 3

### Flu Virus (H9N1) Tests in Mice

Female ICR mice with the weight of 20 to 22 g were used in this study, and H9N1 virus was used as an indicator of the flu virus. The mice were first fed with the composition for influenza prevention and treatment at different dosages for 7 days, and then challenged with H9N1 virus through their eyes and noses. The composition was given to the mice for another 14 days after the virus challenge. The virus control and healthy mice control studies were also conducted at the same time. The detailed information about the eight treatments in this study was as follows:

| Treatment | |
|---|---|
| # | Treatment/Composition dosage |
| 1 | 2.25 mg/mouse/day, the composition of the present invention |
| 2 | 1.03 mg/mouse/day, the composition without immune |
| | enhancer |
| 3 | 1.22 mg/mouse/day, the composition without anti-virus agent |
| 4 | 0.32 mg/mouse/day, the composition of the present invention |
| 5 | 0.145 mg/mouse/day, the composition without immune |
| | enhancer |
| 6 | 0.17 mg/mouse/day, the composition without anti-virus agent |
| 7 | virus control, without feeding the mice with the composition |
| 8 | healthy mice, without H9N1 challenge |

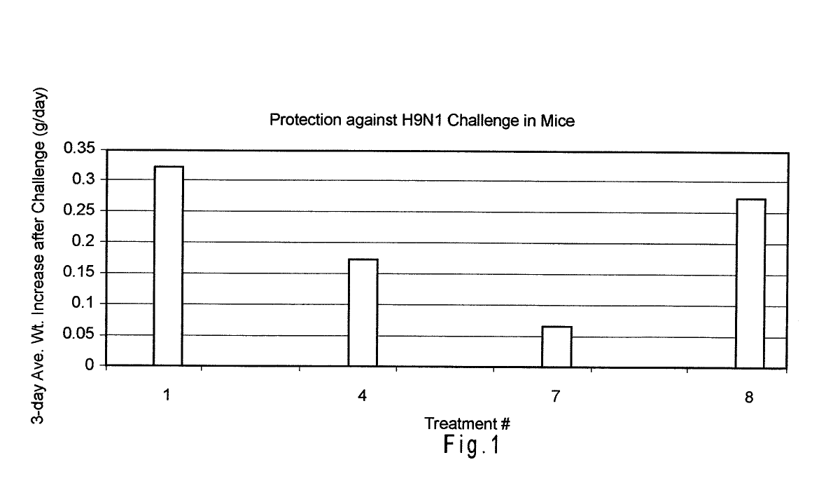

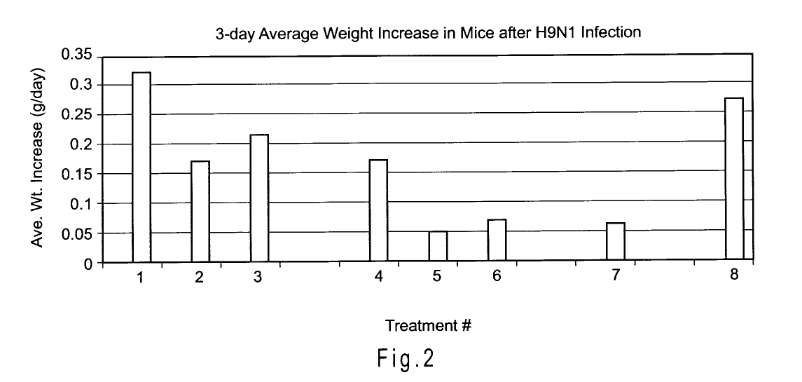

Fifteen mice were used in each treatment. The body weight of each mouse was measured before and after virus challenge. In this experiment, the effectiveness of the composition was determined by the health of the mice, which was measured in terms of the three-day average weight increase—a larger weight increase signifies better health. The efficacy of the components of the composition was also determined, by treating the mice with the composition having and lacking different components. The test results are summarized in FIGS. 1 and 2.

Referring to FIG. 1, the efficacy of the composition was determined. When the mice were not presented the H9N1 challenge, the three-day average weight increases was about 0.27 grams per day. This represented the average weight increase for healthy mice. When the mice were presented the H9N1 challenge and received no treatment, the three-day average weight increase was about 0.06 grams per day. This represented the average weight increase for virus infected mice, and as expected was substantially lowered than that for healthy mice. When the mice were presented the H9N1 challenge and received the composition at 0.32 miligrams per mouse per day, the three-day average weight increase was about 0.17 grams per day. When the mice were presented the H9N1 challenge and received the composition at 2.25 miligrams per mouse per day, the three-day average weight increase was about 0.33 grams per day.

As shown, of the infected mice, those that received the composition demonstrated a higher average weight increase than those that did not. The average weight increased more significantly at a higher concentration of the composition administered. Furthermore, the composition administered at 2.25 miligrams per mouse per day was found to match the average weight increase of the healthy mice. Consequently, the results show that the composition can protect the mice against H9N1 virus challenge.

Referring to FIG. 2, the efficacy of the composition having both the immune enhancer and the anti-viral agent and the composition without the immune enhancer was compared. The conditions for and results of the healthy and infected mice were identical to those as shown in FIG. 1.

When the mice were presented the H9N1 challenge and received the composition at 0.32 miligrams per mouse per day without the immune enhancer, the three-day average weight increase was about 0.05 grams per day. This was substantially lower than that obtained by the composition with the immune enhancer at similar conditions. When the mice were presented the H9N1 challenge and received the composition at 2.25 miligrams per mouse per day without the immune enhancer, the three-day average weight increase was about 0.17 grams per day. This was also substantially lower than that obtained by the composition with the immune enhancer at similar conditions.

Consequently, the composition with both the immune enhancer and anti-viral agent shows superior protection against H9N1 infection than the composition without the immune enhancer. Moreover, at 0.32 miligrams per mouse per day without the immune enhancer, the average weight increase was found to be similar to the infected mice, thus, it can be inferred that the composition without the immune enhancer at low concentration was ineffective to protect against H9N1 infection. At 2.25 miligrams per mouse per day without the immune enhancer, the average weight increase was found to be significantly higher than the infected mice, but was still much lower than the healthy mice, thus suggesting the composition without the immune enhancer cannot sufficiently protect against H9N1 infection.

The efficacy of the composition having both the immune enhancer and the anti-viral agent and the composition without the anti-viral agent was also compared. The conditions for and results of the healthy and infected mice were identical to those as shown in FIG. 1.

When the mice were presented the H9N1 challenge and received the composition at 0.32 miligrams per mouse per day without the anti-viral agent, the three-day average weight increase was about 0.07 grams per day. This was substantially lower than that obtained by the composition with the anti-viral agent at similar conditions. When the mice were presented the H9N1 challenge and received the composition at 2.25 miligrams per mouse per day without the anti-viral agent, the three-day average weight increase was about 0.21 grams per day. This was also substantially lower than that obtained by the composition with the anti-viral agent at similar conditions.

Consequently, the composition with both the immune enhancer and anti-viral agent shows superior protection against H9N1 infection than the composition without the anti-viral agent. Moreover, at 0.32 miligrams per mouse per day without the anti-viral agent, the average weight increase was found to be similar to the infected mice, thus, it can be inferred that the composition without the anti-viral agent at low concentration was ineffective to protect against H9N1 infection. At 2.25 miligrams per mouse per day without the anti-viral agent, the average weight increase was found to be significantly higher than the infected mice, but was still much lower than the healthy mice, thus suggesting the composition without the anti-viral agent cannot sufficiently protect against H9N1 infection.

### Example 4

### In-Vitro Tests—Cytokine (TNF-α) Secretion

The efficacy of the composition and the composition without the immune enhancer was further compared and observed in another in-vitro test as measured by the amount of the TNF-α secretion induced. The filtrates were obtained by filtering the solution of the compositions through 0.22 μm filters.

THP-1 cells (peripheral blood; monocyte; acute monocytic leukemia) were used in the experiment. The cells were cultivated in RPMI 1640 medium for 4-5 days to reach the required cell number. 5×10⁵ of the cells were then seeded into each of the 24-well plate containing RPMI 1640 medium. Triplicate tests were performed for each concentration of the composition. The 24-well plate containing THP-1 cells and the composition filtrates were incubated at 37° C. in a CO₂ incubator for 4 hours. 500 μl of the supernatant in each well was then collected by centrifugation at 2500 rpm for 5 minutes and stored at −80° C. freezer until the assay for TNF-α cytokine was performed. The concentration of TNF-α in the THP-1 cell supernatants was measured by an enzyme linked immunosorbent assay (ELISA) according to the technical data sheet of the BD Biosciences TNF-α testing kit.

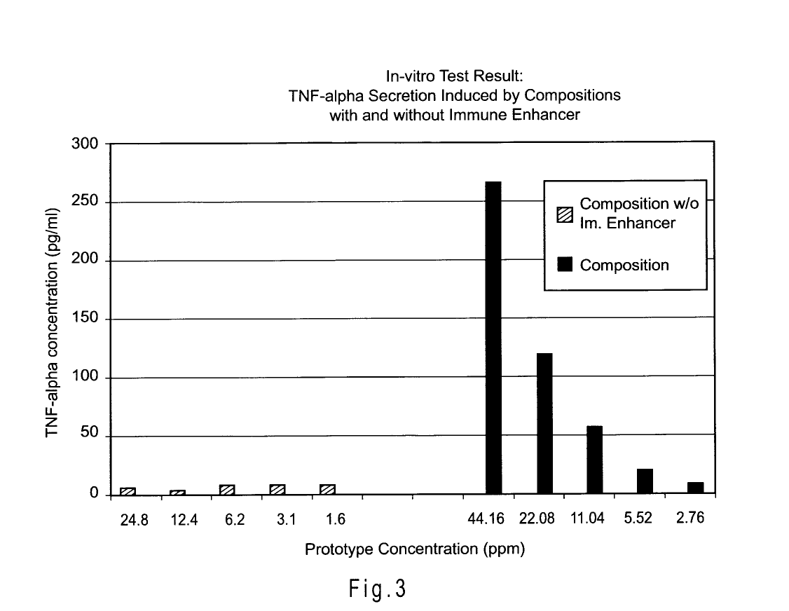

Referring to FIG. 3, across all prototype concentrations, those cells treated by the composition with the immune enhancer produced higher TNF-α concentration than those without the immune enhancer. At higher composition concentrations, the differences were even more pronounced, with the composition substantially out-performing the composition without the immune enhancer.

Consequently, the results show that the composition with the immune enhancer induces more TNF-α secretion than the composition without the immune enhancer, and with higher composition concentration producing a more significant effect.

### Example 5

### Flu Virus (H5N1) Tests in Mice

Female balb/c mice (with weight of 20-22 g) were used in this study. The composition of the present invention was fed at different dosages into the mice for 14 days. On day 8, the mice were infected with avian flu H5N1-A virus through their noses. As a positive control, an anti-flu drug, Adamantane, was given to the mice in the place of the composition. In addition, a saline solution was used in the place of the composition as a negative (virus) control. The mice were observed for 21 days and their activity and death were recorded.

The results were displayed in Table 3. When the mice were presented the H5N1 virus challenge and received no treatment, the mice were dying rapidly; about 80 percent of them had died by day 10 and all of them had died by day 11. This represented the virus control. When the mice were presented the H9N1 virus challenge and received Adamantane at 0.4 miligrams per mouse, the mice were also dying rapidly, although at a slower pace: about 70 percent of them had died by day 11 and 80 percent of them had died by day 13.

When the mice were presented the H5N1 virus challenge and received the composition at 0.2 miligrams and at 1.4 miligrams per mouse, respectively, the mice stopped dying after day 10, and about 60 of them were still alive by day 14. This shows a large improvement over the adamantane treatment. Consequently, the results show that the composition can be effective to counter H5N1 virus.

TABLE 3
Protection against H5N1 Virus Challenge in Mice

| | Percent mouse survival at | | | | | | | | |
|---|---|---|---|---|---|---|---|---|---|
| Treatments | Day 0 | Day 7 | Day 8 | Day 9 | Day 10 | Day 11 | Day 12 | Day 13 | Day 14 |
| 1.4 mg composition | 100 | 90 | 90 | 80 | 60 | 60 | 60 | 60 | 60 |
| per day per mouse | | | | | | | | | |
| 0.2 ppm composition | 100 | 90 | 90 | 60 | 60 | 60 | 60 | 60 | 60 |
| per day per mouse | | | | | | | | | |
| 0.4 mg Adamantane | 100 | 90 | 80 | 60 | 40 | 30 | 30 | 20 | 20 |
| per day per mouse | | | | | | | | | |
| Virus control | 100 | 90 | 80 | 50 | 20 | 0 | 0 | 0 | 0 |

While the examples of the composition for influenza prevention and treatment have been described, it should be understood that the composition not so limited and modifications may be made. The scope of the composition is defined by the appended claims, and all devices that come within the meaning of the claims, either literally or by equivalence, are intended to be embraced therein.

1. A composition for influenza prevention and treatment, comprising:
an immune enhancer; and
an anti-viral agent.

2. The composition of claim 1, wherein said immune enhancer comprises bio-transformed soya.

3. The composition of claim 1, wherein said immune enhancer comprises an immune enhancing ingredient selected from the group consisting of lactoferrin, immunoglobulins, lingzhi, cordyceps, tinospora ingredients, and combinations thereof.

4. The composition of claim 1, wherein said anti-viral agent comprises St. John's wort.

5. The composition of claim 4, wherein said St. John's wort is derived from Hypericum perforatum plant in a form selected from the group consisting of an extract, a purified compound, and a man-made synthesized compound.

6. The composition of claim 4, wherein said St. John's wort comprises 0.3% w/w hypericin.

7. The composition of claim 6, wherein said anti-viral agent comprises from 20 mg to 5,000 mg per daily dosage of said St. John's wort.

8. The composition of claim 6, wherein said anti-viral agent comprises from 100 mg to 2,000 mg per daily dosage of said St. John's wort.

9. The composition of claim 1, wherein said anti-viral agent comprises pseudohypericin.

10. The composition of claim 1, wherein said anti-viral agent comprises an anti-viral drug selected from the group consisting of adamantane, oseltamivir, zanamivir, amantadine and rimantadine.

11. The composition of claim 1, further comprising an inert ingredient selected from the group consisting of a filler and a flavoring agent.

12. The composition of claim 1, wherein the concentration of said immune enhancer in said composition is from 1 to 99 weight percent.

13. The composition of claim 12, wherein the concentration of said immune enhancer in said composition is from 5 to 95 weight percent.

14. The composition of claim 13, wherein the concentration of said immune enhancer in said composition is from 12.5 to 45 weight percent.

15. The composition of claim 1, wherein said anti-viral agent in said composition is from 1 to 99 weight percent.

16. The composition of claim 15, wherein said anti-viral agent in said composition is from 5 to 95 weight percent.

17. The composition of claim 1, further comprising the form selected from the group consisting of a tablet, a capsule and liquid.

18. A method for influenza prevention and treatment, comprising:
administering to a mammal a composition for influenza prevention and treatment,
wherein said composition comprises an immunity enhancer and an anti-viral agent.

19. The method of claim 18, wherein said immune enhancer comprises bio-transformed soya.

20. The method of claim 18, wherein said immune enhancer comprises an immune enhancing ingredient selected from the group consisting of lactoferrin, immunoglobulins, lingzhi, cordyceps, tinospora ingredients, and combinations thereof.

21. The method of claim 18, wherein said anti-viral agent comprises St. John's wort.

22. The method of claim 21, wherein said St. John's wort comprises 0.3% w/w hypericin.

23. The method of claim 22, wherein said anti-viral agent comprises from 20 mg to 5,000 mg per daily dosage of said St. John's wort.

24. The method of claim 22, wherein said anti-viral agent comprises from 100 mg to 2,000 mg per daily dosage of said St. John's wort.

25. The method of claim 21, wherein said St. John's wort is derived from Hypericum perforatum plant in a form selected from the group consisting of an extract, a purified compound, and a man-made synthesized compound.
